(19) United States
(12) Patent Application Publication
(10) Pub. No.: US 2015/0164510 A1
(43) Pub. Date: Jun. 18, 2015

(54) SURGICAL CLIP WITH INNER SPRING

(71) Applicants: Karlsruher Institut Fur Technologie, Karlsruhe (DE); AESCULAP AG, Tuttlingen (DE)

(72) Inventors: Thomas Pleil, Bad Durrheim (DE); Iwiza Tesari, Eggenstein-Leopoldshafen (DE); Klaus-Dieter Steinhilper, Tuttlingen (DE); Markus Nesper, Tuttlingen (DE)

(21) Appl. No.: 14/407,944

(22) PCT Filed: May 28, 2013
(86) PCT No.: PCT/EP2013/060924

§ 371 Date: Dec. 12, 2014

(30) Foreign Application Priority Data
Jun. 29, 2012 (DE) 1020122211379.7

(51) Int. Cl. A61B 17/122

(52) U.S. Cl. CPC A61B 17/1227

(57) ABSTRACT

The present application discloses a surgical clip with two clip branches that each have a clamping portion and an actuating portion, and with a flexion spring arrangement via which the two clip branches are coupled integrally to each other. The flexion spring arrangement has more than a half winding, and an elastic body is arranged in the flexion spring arrangement in such a way that it is deformable by opening the surgical clip.

The present invention relates to a clip with an inner spring.

### PRIOR ART

There are a plurality of approaches in the prior art for controlling or for increasing the closing force of a surgical clip. One simple possible way of increasing the closing force of a surgical clip consists in increasing, in the case of a clip having a leg spring, the number of windings of the leg spring. It is also possible, however, for the cross section of the leg spring to be changed, such that the geometric moment of inertia of the spring increases. The geometric moment of inertia of the leg spring, which is a spring exposed to bending loads, together with the modulus of elasticity of the leg spring determines the profile of the elastic deformation or deflection depending on the applied force or the applied moment.

Owing to the fact that, in the medical sector, the materials used have to be tested thoroughly and approved, only relatively few materials are available. A change in the cross section of the leg spring is expedient primarily when the height of the wound cross section is changed, since the height has a much greater influence on the geometric moment of inertia than the width of this cross section. A change in this cross section also involves disadvantages, however. On the one hand, the region in which the spring can be deformed without plasticization thereof occurring is reduced as a result, and on the other hand the dimensions of the spring are increased as a result. When more windings are used, too, the dimensions of the spring are increased.

Another possible way of increasing the spring force of a surgical clip is to apply a reinforcing clip to the surgical clip. To this end, use is most commonly made of clips with laterally bent branches, which are placed onto the branches of the surgical clip and therefore reinforce the surgical clip. However, this procedure requires more space than an individual clip, involves the risk that the reinforcing clip will become detached from the surgical clip, and makes the process considerably more expensive, firstly since two clips have to be used and secondly since both clips have to be applied in succession.

It is therefore an object of the present invention to provide a surgical clip having an increased spring force, without the outer dimensions thereof being increased.

The object of the present invention is achieved by a surgical clip as claimed in claim 1. Further advantageous embodiments are the subject matter of the dependent claims.

According to one aspect of the present invention, a surgical clip has two clip branches each having a clamping portion and an actuating portion and also a flexible spring arrangement, by way of which the two clip branches are integrally coupled to one another. The two clip branches and the flexible spring arrangement can in this case be produced from one material and one piece, but they can also be produced from different materials, which are then brought together. Moreover, the flexible spring arrangement has more than a half winding, and an elastic body is arranged in the flexible spring arrangement in such a way that it can be deformed by an opening operation of the surgical clip.

If the flexible spring arrangement does not have more than a half winding, it is not possible to arrange an elastic body in the flexible spring arrangement in such a way that it remains securely therein, since it can otherwise emerge or escape at least laterally from the flexible spring arrangement. The clip preferably has one and a half windings, for instance. The elastic body is arranged in the flexible spring arrangement in accordance with the invention when the elastic body is deformed as soon as the surgical clip is opened. If the surgical clip is opened, i.e. the clamping portions are moved apart, the inner diameter of the flexible spring arrangement is reduced. This reduction in the inner diameter of the flexible spring arrangement has an effect on the elastic body, and this leads to deformation of the elastic body and to an increase in the spring force of the surgical clip.

According to an advantageous embodiment of this aspect of the invention, the elastic body is a substantially C-shaped elastic body. This C-shaped body is arranged in the flexible spring arrangement in such a way that it is in contact with the inner face of the flexible spring arrangement substantially along its entire outer face. Here, “substantially along its entire outer face” does not mean that the entire or almost the entire outer face has to be in contact with the inner face of the flexible spring arrangement. In the case of a flexible spring arrangement which is produced from a round profile, contact lines form between the flexible spring arrangement and the C-shaped body. In principle, what is formed is a helical contact line, which is interrupted at the location at which the body is slotted in the axial direction in order to obtain the C shape. Here, “substantially along its entire outer face” therefore means that the elastic body forms contact points with the flexible spring arrangement substantially along its entire circumference.

As an alternative thereto, it is also possible that the outer face of the elastic body comes into contact with the inner face of the flexible spring arrangement only at a few points. In this case, too, the elastic C-shaped body would serve as a spring force reinforcement for the surgical clip. However, in the case of such a design, it may be the case that tissue is accidentally clamped between the flexible spring arrangement and the elastic body, since in this case the distance between the outer face of the elastic body and the inner face of the flexible spring arrangement changes. In principle, in such a case the elastic body has to bear only against at least two points on the inner face of the flexible spring arrangement. If there are only two bearing points or contact points, these have to lie diametrically opposite one another in the flexible spring arrangement. If three or more bearing or contact points are formed, these can be distributed freely along the inner face of the flexible spring arrangement, provided that they extend over an angular range of at least 180°. That is to say, if the first contact point between the elastic body and the flexible spring arrangement is connected to the center of the flexible spring arrangement and the last contact point between the elastic body and the flexible spring arrangement is likewise connected to the center of the flexible spring arrangement, these two connection lines have to include an angle of at least 180°. Otherwise, there would be no clamping action between the elastic body and the flexible spring arrangement, and the elastic body would not serve as a spring reinforcement and would fall out of the inner region of the flexible spring arrangement.

According to another advantageous embodiment of this aspect of the invention, the substantially C-shaped elastic body is open toward the clamping portions of the clip branches. This means that the slot in the lateral face, which distinguishes the C-shaped body from a hollow cylindrical body, branches toward the center of the jaw of the surgical clip.

Alternatively, the slot can also point away from the center of the jaw or can be arranged in any desired direction in relation thereto. The orientation of the slot toward the jaw center allows for a better view in the direction of the jaw and moreover ensures a uniform spring force reinforcement.

According to a further advantageous embodiment of this aspect of the invention, the elastic body is a substantially bar-shaped elastic body, which is arranged in the flexible spring arrangement in such a way that the contact points between the lateral face of the elastic bar-shaped body and the inner face of the flexible spring arrangement are preferably distributed uniformly along the entire circumference of the elastic bar-shaped body. As already explained above for the C-shaped elastic body, this does not mean that the entire lateral face of the elastic body has to be in contact with the flexible spring arrangement, but rather that the contact points between the lateral face of the elastic body and the inner face of the flexible spring arrangement are located substantially along the entire circumference of the elastic body. In particular, a contact point does not have to extend over the entire axial length of the outer face or lateral face of the elastic body.

According to an advantageous embodiment of this aspect of the invention, the substantially bar-shaped elastic body has a circular, a polygonal or a star-shaped cross section. This may be a regular or irregular polygonal or star-shaped cross section. In addition, the bar-shaped elastic body can not only have the cross-sectional shape of a star polygon, but also that of a star which is defined by a circumscribed circle, an inscribed circle and the number of vertices (examples: NATO star, Mercedes star). In the case of a circular cross section, a circumferential contact point is formed between the outer face or lateral face of the elastic body and the inner face of the flexible spring arrangement. In the case of a polygonal or star-shaped cross section, a corresponding number of individual contact points are formed, these being distributed uniformly along the circumference of the elastic body in the case of regular cross sections, and otherwise non-uniformly.

According to another advantageous embodiment of this aspect of the invention, the elastic body is a spring cage, which is arranged in the flexible spring arrangement in such a way that it is in contact with the inner face of the flexible spring arrangement with its outer face substantially along its entire circumference. In this embodiment, contact points are formed between the outer face of the spring cage and the inner face of the flexible spring arrangement only at locations at which a grid bar defines the outer face of the spring cage. The grid bars of the spring cage are preferably distributed uniformly along the circumference thereof.

According to a further advantageous embodiment of this aspect of the invention, a holding device is provided on at least one end face of the elastic body and protrudes outward beyond the inner radius of the flexible spring arrangement. A holding device of this type can ensure that the elastic body cannot unintentionally escape from the inner region of the flexible spring arrangement. The holding device consists, for example, of two protrusions, which are provided lying diametrically opposite one another on the outer edge of the end face of the elastic body. As an alternative thereto, it is also possible, however, to provide three or more protrusions, which are located in an angular range of at least 180° along the outer edge of the end face.

It is preferred that a holding device is provided on both end faces of the elastic body. By way of example, a holding device can be formed integrally with the elastic body or can be fastened to the elastic body before the latter is arranged in the flexible spring arrangement. The other holding device or else both holding devices is/are then fitted to the elastic body by adhesive bonding, screwing, welding or by another method once the elastic body has been arranged in the flexible spring arrangement. The distance between the holding device and the side faces of the flexible spring arrangement is preferably small. A certain gap between the holding device and the side faces of the flexible spring arrangement should, however, be retained, so as not to generate any undesirable friction between these components.

According to an advantageous embodiment of this aspect of the invention, the holding device extends substantially along the entire outer circumference of the elastic body. The holding device is so to speak an annular element which forms a type of collar. The collar or the holding device does not have to extend along the entire circumference of the flexible spring arrangement, however. In the case of a C-shaped elastic body, the holding device preferably extends along the entire end face of the C-shaped body. However, the holding device can also be located only at specific portions of the end face of the elastic body, for example in the region of both ends and of the central portion of the C-shaped elastic body.

According to a further particularly advantageous embodiment of this aspect of the invention, the elastic body has a variable cross section in the axial direction, in such a manner that, on both sides of the flexible spring arrangement, it has a circumscribed circle which is enlarged compared to the central region, at which it is in contact with the inner face of the flexible spring arrangement. This means that the elastic body forms a substantially circumferential groove in the region in which the flexible spring arrangement is located. The flexible spring arrangement is located at least partially in said groove and the inner face of the flexible spring arrangement is at least partially in contact with the base face of the circumferential groove. In the case of a C-shaped elastic body, this can likewise be provided with a groove. To introduce this C-shaped body into the flexible spring arrangement, the C-shaped body only has to be bent together slightly further than is the case in the basic position of the surgical clip. Then, it is pushed laterally into the flexible spring arrangement and relaxed, as a result of which it bears against the inner face of the flexible spring arrangement. If the C-shaped elastic body is positioned correctly, the flexible spring arrangement latches into the circumferential groove of the C-shaped body and thereby secures the position thereof in the flexible spring arrangement.

According to a particular embodiment of this elastic body with an axially variable cross section, a groove can be formed in such a manner that it forms a notch-like recess in the outer circumferential face of the elastic body. A V-shaped circumferential groove is therefore created in this case. In this case, what is created is a self-centering C-shaped elastic body, the circumferential groove of which has two oblique faces. Since the C-shaped elastic body is prestressed to a certain degree, at least if the flexible spring arrangement bears against the end region thereof, the oblique faces ensure that the C-shaped elastic body is self-centered with respect to the flexible spring arrangement, since it has a smaller prestress in this position, i.e. if the flexible spring arrangement rests in the central region of the C-shaped elastic body, than if the flexible spring arrangement rests in an end region of the C-shaped elastic body. However, the groove can also be U-shaped, or a substantially V-shaped groove with curved side walls can be created. It is also possible for a step to be formed between the base face of the circumferential groove and the lateral face of the elastic body.

According to a particularly advantageous embodiment of this aspect of the invention, the flexible spring arrangement has one and a half windings. One and a half windings are particularly advantageous since firstly they ensure that the elastic body can come into contact with the inner face of the flexible spring arrangement along its entire circumference, and secondly they do not excessively increase the width of the flexible spring arrangement.

According to a further particularly advantageous embodiment of this aspect of the invention, the two clip branches are crossed between the respective clamping portion and the respective actuating portion. This means that the flexible spring arrangement consists of an odd number of half windings ([2k+1/2, where k is a non-negative integer), and the jaw of the surgical clip is opened by the number of windings being increased tendentially, i.e. by the flexible spring arrangement being wound in further. In this case, the inner diameter of the flexible spring arrangement is reduced, as a result of which an annular compressive force is applied to the elastic body in the flexible spring arrangement.

According to another advantageous embodiment of this aspect of the invention, one of the two clip branches is slotted in the region of the crossing and the other one of the two clip branches extends through said slot and is guided through the slot. In this way, it is possible to prevent a situation in which the spring load is introduced eccentrically into the clamping portions and has the effect that the two clamping portions move past one another beyond the basic position, and therefore reliable closing of the surgical clip cannot be ensured.

According to an advantageous embodiment of this aspect of the invention, the clip is coated or painted at least in the region of the flexible spring arrangement in order to reduce the friction between the flexible spring arrangement and the elastic body. In this way, it is also possible, however, to reduce the friction within the individual windings of the flexible spring arrangement or else also the friction between the flexible spring arrangement and an application instrument, which grips the surgical clip on the flexible spring arrangement and opens it by pressing the flexible spring arrangement together.

According to a further advantageous embodiment of this aspect of the invention, the elastic body is formed from plastic or metal. The elastic body is preferably formed from spring steel or a titanium alloy, if it is a C-shaped elastic body or a grid body. In the case of a bar-shaped elastic body, it is also possible to select a solid rubber material (for example silicone), irrespective of whether the cross section is selected to be circular, polygonal or star-shaped. It goes without saying that it is also possible to use material combinations, for example a metallic C-shaped body with an annular holding device formed from plastic.

Further advantages and features of the invention are evident to a person skilled in the art from the accompanying figures and the detailed description of the exemplary embodiments.

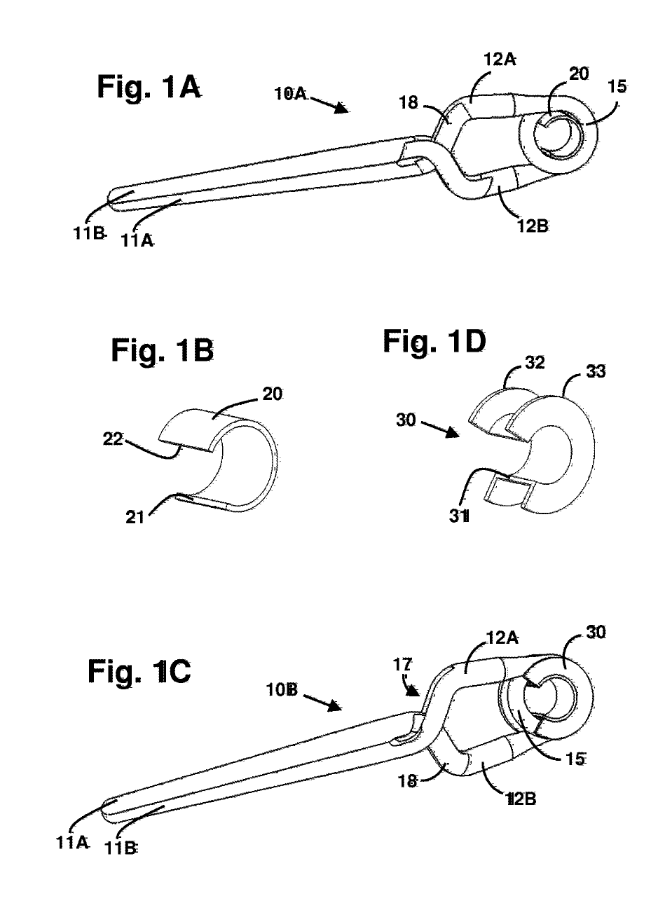

FIG. 1A is a lateral depiction of a first exemplary embodiment of the present invention;

FIG. 1B is an illustration of a C-shaped elastic body corresponding to FIG. 1A;

FIG. 1C is a lateral depiction of a second exemplary embodiment of the present invention;

FIG. 1D is an illustration of a C-shaped elastic body corresponding to FIG. 1C;

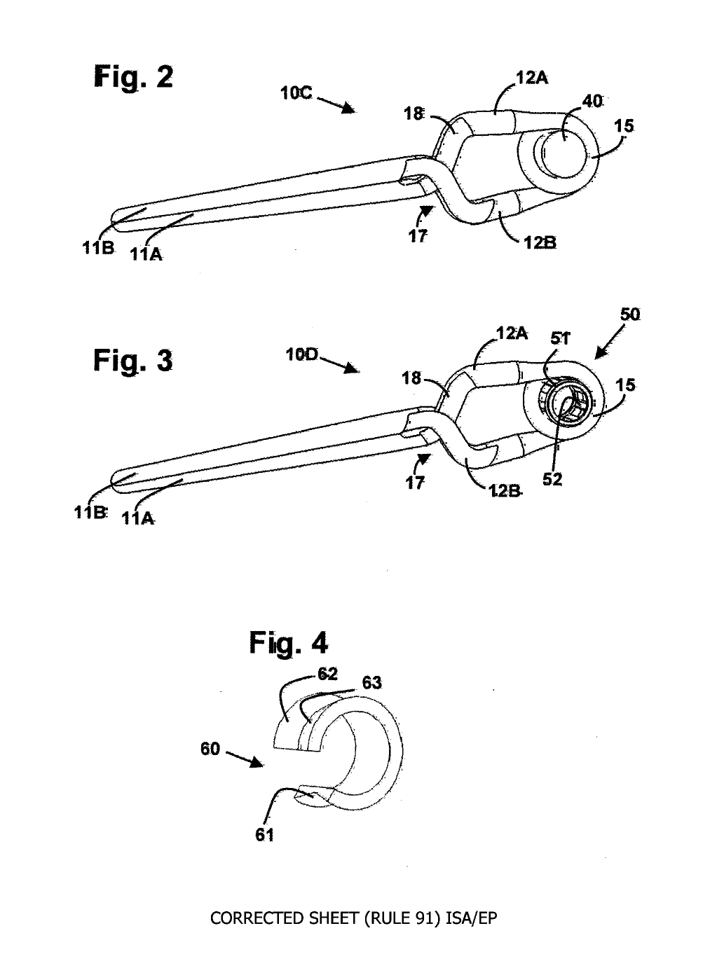

FIG. 2 is a lateral depiction of a third exemplary embodiment of the present invention;

FIG. 3 is a lateral depiction of a fourth exemplary embodiment of the present invention; and

FIG. 4 is an illustration of a C-shaped elastic body according to a fifth exemplary embodiment.

A first exemplary embodiment of the present invention is described in detail hereinbelow with reference to FIGS. 1A and 1B.

The surgical clip 10A according to the first exemplary embodiment has two clip branches. Each clip branch has a clamping portion 11A, 11B and an actuating portion 12A, 12B. The surgical clip 10A moreover has a flexible spring arrangement 15, by way of which the two clip branches are integrally coupled to one another.

The flexible spring arrangement 15 of this exemplary embodiment has one and a half windings, and a C-shaped elastic body 20, which is shown in detail in FIG. 1B, is arranged in the flexible spring arrangement 15. The C-shaped elastic body 20 bears with its entire outer circumferential face 21 against the inner circumferential face of the flexible spring arrangement 15. The C-shaped elastic body 20 is arranged in such a way that it is open toward the clamping portions 11A, 11B, i.e. the slot in the C-shaped body 20 points toward the jaw center.

The C-shaped elastic body 20 moreover has a uniform thickness along its circumference. In another embodiment, the thickness of the elastic body 21 can also be different. The elastic body 20 can be formed from various materials, for example spring steel, plastic or a titanium alloy. In the present case, it is formed from a titanium alloy, like the entire surgical clip 10A.

In the case of the surgical clip 10A according to this exemplary embodiment, the two clip branches are crossed between the respective clamping portion 11A, 11B and the respective actuating portion 12A, 12B. Here, the clamping portion 11A adjoins the actuating portion 12A and the clamping portion 11B adjoins the actuating portion 12B. A portion 18 of reduced width is formed between the actuating portion 12A and the clamping portion 11A. A slotted portion 17 is formed between the actuating portion 12B and the clamping portion 11B. The portion 18 of reduced width and the slotted portion are crossed with one another, the portion 18 of reduced width extending through the slotted portion 17. In this way, the two clamping portions 11A and 11B are guided in relation to one another. In addition, the flexible spring arrangement 15 has a coating. In the region of the transition from the flexible spring arrangement 15 to the two actuating portions 12A and 12B, provision is made of thickened sections, which are intended to simplify gripping with an applicator instrument.

The C-shaped elastic body 20 of this exemplary embodiment does not have a groove or holding device. It is introduced or inserted into the flexible spring arrangement 15 by being pressed together or rolled together, by being introduced laterally into the inner space of the flexible spring arrangement 15 and by then being relaxed. By virtue of the fact that it is not completely relaxed even in the basic position of the surgical clip 10A, it is clamped firmly on the inner face of the flexible spring arrangement 15.

The surgical clip 10A according to this exemplary embodiment is opened by the two actuating portions 12A, 12B being moved toward one another. Since the two clip branches are crossed, the two clamping portions 11A, 11B move apart as a result. The inner diameter of the flexible spring arrangement 15 is reduced as a result, and this has the effect that the C-shaped elastic body 20 is bent further, producing an additional load on the flexible spring arrangement 15 and increasing the total clamping power of the surgical clip 10A. The elastic C-shaped body 20 acts like a bent leaf spring, which is connected in parallel with the flexible spring arrangement 15 and supports it. In this exemplary embodiment, the inner faces of the clamping portions 11A, 11B are formed in an atraumatic manner.

The gap between the two ends 21, 22 of the C-shaped elastic body 20 is chosen in such a way that the two ends 21, 22 do not butt against one another during the opening operation; this would lead to a limitation of the opening angle of the clip 10A. In the present exemplary embodiment, the opening angle is limited by the geometry of the slotted portion 17 and of the portion of reduced width 18. If there is no such opening angle limitation and it is feared that the flexible spring arrangement 15 might plasticize on account of excessive opening of the jaw, which has the effect that the jaw no longer closes completely or at least no longer closes with the full load, the C-shaped elastic body 20 with its two ends 21, 22 can be assessed as an opening angle limitation.

A second exemplary embodiment of the present invention is described in detail with reference to FIGS. 1C and 1D. Since the surgical clip 10B has a similar structure to the surgical clip 10A according to the first exemplary embodiment, details are provided here predominantly in relation to the differences with respect to the first exemplary embodiment; the features which are not discussed are identical to those in the first exemplary embodiment.

Instead of a simple C-shaped elastic body 20, in this exemplary embodiment provision is made of a C-shaped elastic body 30 with a holding device 32 and 33 on each of the end faces thereof. In this exemplary embodiment, the two holding devices 32, 33 each extend along the entire circumferential face 31 of the C-shaped elastic body 30. The two holding devices 32, 33 therefore each form a type of collar, which protrudes outward from the elastic C-shaped body 30 to such an extent that the flexible spring portion 15 of the surgical clip 10B is received completely between the two collars 23, 33.

The collar 32 is in this case formed integrally with the C-shaped elastic body 31 in that a collar formed from plastic is molded onto a metallic C-shaped body 31. This component is then inserted into the flexible spring arrangement 15, and then the second collar 32 is fitted to the C-shaped body 31. In the present case, the collar 32, which is likewise formed from plastic, is pushed onto the C-shaped body 31. In this case, the inner diameter of the collar or of the holding device 32 has a certain undersize, in order to ensure the bond to the body 31.

If the same material is used for the holding devices 32, 33 and for the elastic body 31, the components can also be welded effectively. In particular, this can be accomplished effectively in the case of metallic components or plastic components.

In an embodiment according to the present exemplary embodiment, the dimensions and the material of the holding devices 32, 33 have a major influence on the elasticity or the bending behavior of the respective elastic body 30, 40, 50. In order to reduce the influence that the holding devices 32, 33 have on the bending behavior of the elastic body 30, 40, 50, the holding devices 32, 33 can be slotted from the outside.

Since, in this exemplary embodiment, provision is made of two holding devices which secure the elastic body 30 in the flexible spring arrangement 15, the elastic body 30 or the C-shaped body 31 can be dimensioned in such a way that it is completely relaxed in the basic position of the surgical clip 10B, as is shown in FIG. 1B. The elastic body 30 can therefore be dimensioned in such a way that either it applies a load to the flexible spring arrangement 15 already in the basic position of the clip 10B, it applies a load immediately to the flexible spring arrangement 15 when the clamping portions 11A, 11B are moved out of the basic position, or it applies a load to the flexible spring arrangement only after a certain displacement of the clamping portions 11A, 11B out of the basic position. In this way, it is possible to set the displacement/force behavior of the jaw opening of the clip 10B.

A third exemplary embodiment of the present invention is described in detail hereinbelow with reference to FIG. 2. In this exemplary embodiment, an elastic body is provided in the flexible spring arrangement 15. The elastic body of this exemplary embodiment consists of a solid body made of rubber, but this can also be a solid body made of a composite material, for example a layer structure made up of rubber and metal. This composite material can either be made up of planar layers or can consist of a plurality of metallic tubes which are pushed one into another and are cast with rubber. In principle, any form of composite material can be used, but an approximately isotropic material is generally to be preferred to an orthotropic material, since in the case of an isotropic material it is not necessary to pay attention to the position thereof in the flexible spring arrangement 15.

In this exemplary embodiment, the elastic bar-shaped body 40 is in contact with the inner face of the flexible spring arrangement 15 along its entire circumference. In addition, a groove (not shown in FIG. 2) is formed along the circumference of the elastic body 40 and ensures that the elastic body 40 also does not fall out of the flexible spring arrangement 15 in the basic position of the clip 10C.

It goes without saying that this elastic body 40 can also be provided with one or two holding devices. Here, by way of example, these can consist of two circular plates which are fitted or fastened to the end faces of the body 40.

A fourth exemplary embodiment of the present invention is described in detail hereinbelow with reference to FIG. 3. In the fourth exemplary embodiment, as compared with the third exemplary embodiment, provision is made of a spring cage 50 instead of a bar-shaped elastic body 40.

The spring cage consists of axial bars 52, which are fastened on both sides of the flexible spring arrangement 15 to a support component 51. The elastic action of the spring cage 50 is achieved primarily by virtue of the fact that the axial bars (axial relates here to the axial direction of the spring cage) can bend toward the axis of the spring cage. If a more rigid spring cage 50 is required, more axial bars 52 can be provided, the bars 52 themselves can be provided with a more rigid form or provision can be made of additional annular components, which connect the individual bars 52 to one another in the region of the flexible spring arrangement 15. However, the spring cage 50 can also be cast, for example, with a plastic material such as, for example, a rubber in accordance with the third exemplary embodiment, in order to increase the modulus of elasticity. If the support components 51 have a diameter greater than the inner diameter of the flexible spring arrangement 15, they simultaneously serve as holding devices.

An elastic C-shaped ring according to a fifth exemplary embodiment is described in detail hereinbelow with reference to FIG. 4.

The C-shaped elastic body 60 according to this exemplary embodiment has a circumferential V-shaped groove 61. If the body is arranged in a flexible spring arrangement, the prestress of the C-shaped body ensures, in cooperation with the inclined or oblique faces 62, 63, that the elastic body 60 is centered in relation to the flexible spring arrangement 15 in the axial direction. In this way, it can easily be ensured that the C-shaped body 60 is positioned correctly.

Many features which are described here in relation to the individual exemplary embodiments can also be implemented, however, in combination with other exemplary embodiments, e.g. a wide variety of holding devices 32, 33 can be combined with a wide variety of elastic bodies 20, 30, 40, 50, 60. Similarly, the materials indicated can be used for all exemplary embodiments. As a whole, all features of the individual exemplary embodiments can be combined in a suitable manner as desired.

1. A surgical clip with two clip branches each having a clamping portion and an actuating portion and also with a flexible spring arrangement, by way of which the two clip branches are integrally coupled to one another, wherein the flexible spring arrangement has more than a half winding, and an elastic body is arranged in the flexible spring arrangement in such a way that it can be deformed by an opening operation of the surgical clip, and three or more contact points are formed between the elastic body and the flexible spring arrangement.

2. The surgical clip as claimed in claim 1, wherein
the elastic body is a substantially C-shaped elastic body, which is arranged in the flexible spring arrangement in such a way that it is in contact with the inner face of the flexible spring arrangement substantially along its entire outer face.

3. The surgical clip as claimed in claim 2, wherein
the substantially C-shaped elastic body is open toward the clamping portions of the clip branches.

4. The surgical clip as claimed in claim 2, wherein
the substantially C-shaped elastic body has a circumferential groove on its outer circumferential face, said groove being adapted to at least partially receive the flexible spring arrangement.

5. The surgical clip as claimed in claim 1, wherein
the elastic body is a substantially bar-shaped elastic body, which is arranged in the flexible spring arrangement in such a way that the contact points between the lateral face of the elastic bar-shaped body and the inner face of the flexible spring arrangement are preferably distributed uniformly along the entire circumference of the elastic bar-shaped body.

6. The surgical clip as claimed in claim 5, wherein
the substantially bar-shaped elastic body has a circular, a polygonal or a star-shaped cross section.

7. The surgical clip as claimed in claim 2,
wherein
the elastic body is a spring cage, which is arranged in the flexible spring arrangement in such a way that it is in contact with the inner face of the flexible spring arrangement with its outer face substantially along its entire circumference.

8. The surgical clip as claimed in claim 1,
wherein
a holding device is provided on at least one end face of the elastic body and protrudes outward beyond an inner radius of the flexible spring arrangement.

9. The surgical clip as claimed in claim 8, wherein
the holding device extends substantially along the entire outer circumference of the elastic body.

10. The surgical clip as claimed claim 2,
wherein
the elastic body has a variable cross section in an axial direction, in such a manner that, on both sides of the flexible spring arrangement, it has a circumscribed circle which is enlarged compared to a central region, at which it is in contact with the inner face of the flexible spring arrangement.

11. The surgical clip as claimed in claim 1,
wherein
the flexible spring arrangement has one and a half windings.

12. The surgical clip as claimed in claim 1,
wherein
the two clip branches are crossed between the respective clamping portion and the respective actuating portion.

13. The surgical clip as claimed in claim 12, wherein
one of the two clip branches is slotted in the region of the crossing and the other one of the two clip branches extends through said slot and is guided through the slot.

14. The surgical clip as claimed in claim 1, wherein
the clip is coated or painted at least in a region of the flexible spring arrangement in order to reduce the friction between the flexible spring arrangement and the elastic body.

15. The surgical clip as claimed in claim 1, wherein
the elastic body is formed from plastic or metal, preferably from spring steel or a titanium alloy.
